(19) United States
(12) Patent Application Publication
(10) Pub. No.: US 2005/0277205 A1
(43) Pub. Date: Dec. 15, 2005

(54) Multicomponent magnetic nanorods for biomolecular separations

(76) Inventors: Ki-Bum Lee, San Diego, CA (US); Sungho Park, Evanston, IL (US); Chad A. Mirkin, Wilmette, IL (US); Byung-Keun Oh, Evanston, IL (US)

Correspondence Address: David A. Casimir;MEDLEN & CARROLL, LLP, Suite 350, 101 Howard Street, San Francisco, CA, 94105, US

(21) Appl. No.: 11/062,983

(22) Filed: Feb. 22, 2005

Related U.S. Application Data
(60) Provisional application No. 60/546,641, filed on Feb. 20, 2004.

(51) Int. Cl. C12Q 1/68; G01N 33/553

(52) U.S. Cl. 436/526

(57) ABSTRACT

The present invention relates to methods and compositions for separation of proteins. In particular, the present invention provides multicomponent nanorods for biomolecular separations of proteins.

This application claims priority to provisional patent application Ser. No. 60/546,641 filed Feb. 20, 2004, which is herein incorporated by reference in its entirety.

This invention was made with government support under Air Force Office of Scientific Research (AFOSR) grant F49620-00-1-0283. The government has certain rights in the invention.

### FIELD OF THE INVENTION

The present invention relates to methods and compositions for separation of proteins and other molecules. In particular, the present invention provides multicomponent nanorods for biomolecular separations of proteins and other molecules.

### BACKGROUND OF THE INVENTION

As the nucleic acid sequences of a number of genomes, including the human genome, become available, there is an increasing need to interpret this wealth of information. While the availability of nucleic acid sequence allows for the prediction and identification of genes, it does not explain the expression patterns of the proteins produced from these genes. The genome does not describe the dynamic processes on the protein level. For example, the identity of genes and the level of gene expression does not represent the amount of active protein in a cell nor does the gene sequence describe post-translational modifications that are essential for the function and activity of proteins. Thus, in parallel with the genome projects there has begun an attempt to understand the proteome (i.e., the quantitative protein expression pattern of a genome under defined conditions) of various cells, tissues, and species. Proteome research seeks to identify targets for drug discovery and development and provide information for diagnostics (e.g., tumor markers and protein profiles).

An important area of proteomics research is the purification of recombinant proteins of interest. These purified proteins are employed for numerous purposes such as for example, the preparation of targets for drug discovery and antigens in the preparation of antibodies used, in turn, as diagnostic reagents and therapeutic agents. In order to easily purify a recombinant protein, fusion proteins comprising a protein of interest fused to a “tag” are often employed. The fusion protein is expressed in a cell and is then contacted with a material, such as a chromatography resin, that specifically interacts with and binds to the tag. This permits the separation and recovery of purified fusion protein from complex mixtures. However, these purification methods are often time consuming and laborious. What is needed in the art are efficient methods for the purification of proteins of interest.

### SUMMARY OF THE INVENTION

The present invention relates to methods and compositions for separation of proteins and other molecules. In particular, the present invention provides multicomponent nanorods for biomolecular separations of proteins and other molecules.

For example, in some embodiments, the present invention provides mulitcomponent nanorods comprising one or more (e.g., two or more) binding domains. In some embodiments, the binding domains comprise metals that serve as binding domains for protein affinity tags. In preferred embodiments, the nanorods are magnetic. In some embodiments, the present invention provides methods of purifying proteins or other molecules of interest using the nanorods. In some embodiments, two or more proteins of interest are fused to protein affinity tags that are specific for different binding domains of the nanorods. The fusion proteins are contacted with the nanorods, the nanorods are separated from the rest of the solution using a magnetic field, and the fusion proteins are selectively eluted from the nanorods. By altering the solution conditions, the fusion proteins can be separately eluted from the nanorods, allowing for the simultaneous purification of two or more proteins or other molecules of interest.

Accordingly, in some embodiments, the present invention provides a composition comprising a plurality of multifunctional nanorods, wherein the nanorods comprise one or more (e.g., 2 or more, and preferably three or more) binding domains. In some embodiment, the two or more binding domains comprise a first binding domain that is configured to bind a first protein affinity tag and a second binding domain that is configured to bind a second protein affinity tag. In some embodiments, the first protein affinity tag is one or more histidines. In some embodiments, the first binding domain comprises nickel. In some embodiments, the second binding domain comprises gold. In some embodiments, the second protein affinity tag is biotin. In some embodiments, the second binding domain comprises nitrated streptavidin. In certain embodiments, the nitrated streptavidin is attached to the gold of the nanorod. In some embodiments, the protein affinity tags are covalently attached to proteins of interest (e.g., wherein the first protein affinity tag is covalently attached to a first protein of interest and a second protein affinity tag is covalently attached to a second protein of interest, and wherein the first and second proteins of interest are different from each other). In some embodiments, the first protein affinity tag and the first protein of interest are present as a first fusion protein and the second protein affinity tag and the second protein of interest are present as a second fusion protein. In some preferred embodiments, the nanorods are configured to be attracted to a magnetic field.

The present invention further provides a method, comprising: providing a plurality of multifunctional nanorods, wherein the nanorods comprise one or more (e.g., 2 or more and preferably three or more) binding domains. In some embodiments, the two or more binding domains comprise a first binding domain that is configured to bind a first protein affinity tag and a second binding domain that is configured to bind a second protein affinity tag; and contacting the nanorods with the first and second protein affinity tags under conditions such that the first protein affinity tag binds to the first binding domain of the nanorods and second protein affinity tag binds to the second binding domain of the nanorods. In some embodiments, the method further comprises the steps of eluting the first protein affinity tag from the nanorod under conditions that the second ligand is not eluted from the nanorod; and eluting the second protein affinity tag from the nanorod. In some embodiments, the first protein affinity tag is one or more histidines. In some embodiments, the first binding domain comprises nickel. In some embodiments, the second binding domain comprises gold. In some embodiments, the second protein affinity tag is biotin. In some embodiments, the second binding domain comprises nitrated streptavidin. In certain embodiments, the nitrated streptavidin is attached to gold of the nanorod. In some embodiments, the protein affinity tags are covalently attached to proteins of interest (e.g., wherein the first protein affinity tag is covalently attached to a first protein of interest and a second protein affinity tag is covalently attached to a second protein of interest, and wherein the first and second proteins of interest are different from each other). In some embodiments, the first protein affinity tag and the first protein of interest are present as a first fusion protein and the second protein affinity tag and the second protein of interest are present as a second protein affinity tag. In some preferred embodiments, the nanorods are configured to be attracted to a magnetic field.

The present invention additionally provides a kit, comprising: a plurality of multifunctional nanorods, wherein the nanorods comprise one or more (e.g. 2 or more and preferably three or more binding domains. In some embodiments, the two or more binding domains comprise a first binding domain and a second binding domain. In some embodiments, the kits further comprise two or more protein affinity tags, wherein a first protein affinity tag is configured to bind the first binding domain and a second protein affinity tag is configured to bind the second binding domain. In some embodiments, the first protein affinity tag is one or more histidines. In some embodiments, the first binding domain comprises nickel. In some embodiments, the second binding domain comprises gold. In some embodiments, the second protein affinity tag is biotin. In some embodiments, the second binding domain comprises nitrated streptavidin. In certain embodiments, the nitrated streptavidin is attached to gold of the nanorod. In some embodiments, the protein affinity tags are covalently attached to proteins of interest (e.g., wherein the first protein affinity tag is covalently attached to a first protein of interest and a second protein affinity tag is covalently attached to a second protein of interest, and wherein the first and second proteins of interest are different from each other). In some embodiments, the first protein affinity tag and the first protein of interest are present as a first fusion protein and the second protein affinity tag and the second protein of interest are present as a second protein affinity tag. In some preferred embodiments, the nanorods are configured to align in a magnetic field. In some embodiments, the protein affinity tags are nucleic acids encoding the protein affinity tags and wherein the nucleic acids are in an expression vector.

### DESCRIPTION OF THE FIGURES

FIG. 1 shows fluorescence spectra before and after separation of poly-His with Au—Ni—Au rods. Inset shows the standard curve and the arrows represent each concentration of poly-His before and after separation.

FIG. 2 shows fluorescence spectra before and after separation of proteins with and without His tags.

FIG. 3 shows fluorescence spectra before and after separation of a mixture of Alexa-488-labeled anti-human IgG and Alexa-568-labeled anti-polyHis IgG with poly-His decorated AuNiAu rods. Black solid lines represent a fluorescence spectrum of a mixture of Alexa-488-labeled anti-human IgG and Alexa-568 labeled anti-polyHis IgG. Dashed black traces show a spectrum of supernatant after separation of Alexa-568-labeled antipolyHis IgG with poly-His decorated Au—NiAu rods. Dashed red traces show a spectrum of supernatant after separation of Alexa568-labeled anti-polyHis IgG from rods by changing pH from 7.4 to 2.8 with an eluent buffer solution.

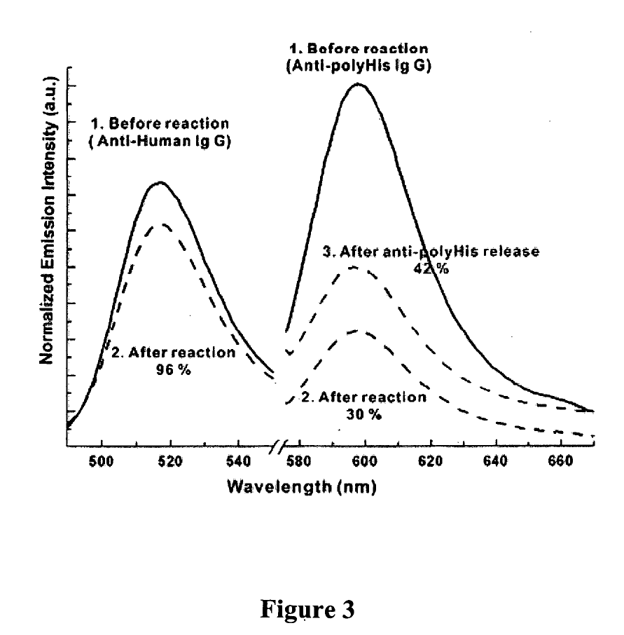

FIG. 4 shows pictures of a suspension of Au—Ni—Au rods in PBS puffer solution (pH=7.4). Au—Ni—Au rods (A) after rigorous shaking, (B) adsorbed to the sidewalls of a vial due to an outside magnetic field.

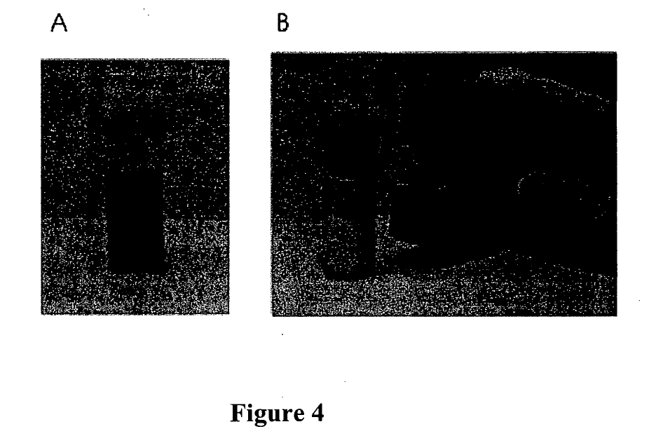

FIG. 5 shows a schematic of protein purification using a biotin-streptavidin system.

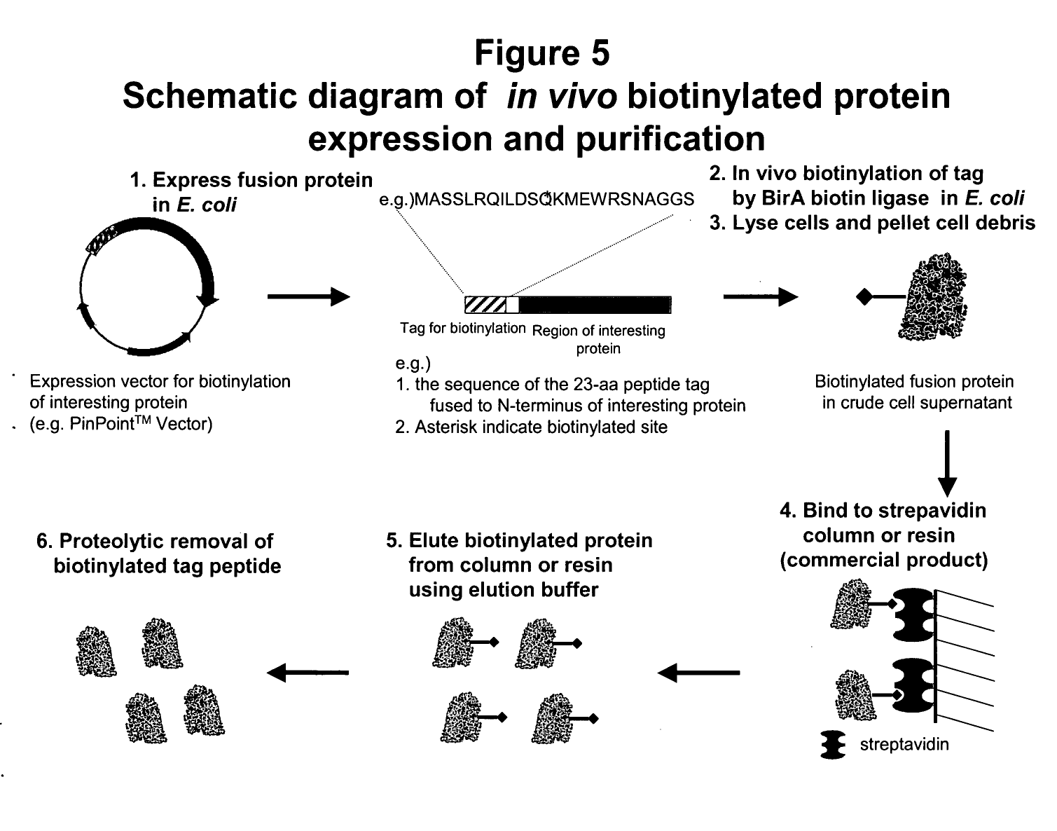

FIG. 6 shows a diagram of preparation of nanorods used in some embodiments of the present invention.

FIG. 7 shows a schematic of one exemplary embodiment of protein purification using the methods of the present invention.

FIG. 8 shows a fluorescence spectrum of nanorods with polyhistidine attached and released.

FIG. 9 shows a fluorescence spectrum of nanorods with biotin tagged proteins attached and released.

FIG. 10 shows a diagram of one exemplary embodiment of protein purification using the methods of the present invention.

### DEFINITIONS

To facilitate an understanding of the present invention, a number of terms and phrases are defined below:

As used herein, the term “multifunctional nanorod” refers to a nanorod or other small (e.g., less than 10 μm and preferably less than 1 μm in any one dimension) support that contains multiple functional or binding domains.

As used herein, the term “protein affinity tag” refers to a molecule (e.g., protein, peptide, carbohydrate, etc.) that associates with a molecule of interest (e.g., protein, peptide) and that further is able to specifically bind, directly or indirectly, to a binding domain of a nanorod. In some embodiments, protein affinity tags are generated by expressing nucleic acids encoding the protein affinity tag (and optionally the molecule of interest).

As used herein, the term “fusion protein” refers to a single polypeptide chain comprising two or more distinct domain (i.e., two or more segments of proteins or peptides combined in a manner not found in nature; e.g., chimeric proteins, purification tags attached to proteins, etc.). In some embodiments, the domains are present sequentially without any intervening amino acids. In other embodiments, the domains are separated by short stretches of amino acids (e.g., linkers).

As used herein, the term “binding domain,” as in “first binding domain” and second binding domain” refers to domains on a nanorod that specifically bind to a protein affinity tag. In some embodiments, the domains are spatially separated. Exemplary protein affinity tag and binding domain pairs include, but are not limited to histidine/nickel and gold/streptavidin/biotin.

As used herein, the term “gene transfer system” refers to any means of delivering a composition comprising a nucleic acid sequence to a cell or tissue. For example, gene transfer systems include, but are not limited to, vectors (e.g., retroviral, adenoviral, adeno-associated viral, and other nucleic acid-based delivery systems), microinjection of naked nucleic acid, polymer-based delivery systems (e.g., liposome-based and metallic particle-based systems), biolistic injection, and the like. As used herein, the term “viral gene transfer system” refers to gene transfer systems comprising viral elements (e.g., intact viruses, modified viruses and viral components such as nucleic acids or proteins) to facilitate delivery of the sample to a desired cell or tissue. As used herein, the term “adenovirus gene transfer system” refers to gene transfer systems comprising intact or altered viruses belonging to the family Adenoviridae.

As used herein, the term “site-specific recombination target sequences” refers to nucleic acid sequences that provide recognition sequences for recombination factors and the location where recombination takes place.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the MRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The MRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed).

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of MRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer or shorter polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

As used herein the term “portion” when in reference to a nucleotide sequence (as in “a portion of a given nucleotide sequence”) refers to fragments of that sequence. The fragments may range in size from four nucleotides to the entire nucleotide sequence minus one nucleotide (10 nucleotides, 20, 30, 40, 50, 100, 200, etc.).

As used herein, the terms “restriction endonucleases” and “restriction enzymes” refer to bacterial enzymes, each of which cut double-stranded DNA at or near a specific nucleotide sequence.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

“Amino acid sequence” and terms such as “polypeptide” or “protein” are not meant to limit the amino acid sequence to the complete, native amino acid sequence associated with the recited protein molecule.

As used herein, the term “protein of interest” encompasses any protein, native or non-native, or variant.

The term “native protein” as used herein to indicate that a protein does not contain amino acid residues encoded by vector sequences; that is, the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four amino acid residues to the entire amino acid sequence minus one amino acid.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher (or greater) than that observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, Virol., 52:456 [1973]), has been modified by several groups to optimize conditions for particular types of cells. The art is well aware of these numerous modifications.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The terms “test compound” and “candidate compound” refer to any chemical entity, pharmaceutical, drug, and the like that is a candidate for use to treat or prevent a disease, illness, sickness, or disorder of bodily function (e.g., cancer). Test compounds comprise both known and potential therapeutic compounds. A test compound can be determined to be therapeutic by screening using the screening methods of the present invention. In some embodiments of the present invention, test compounds include antisense compounds.

As used herein, the term “sample” is used in its broadest sense. In one sense it can refer to a cell lysate. In another sense, it is meant to include a specimen or culture obtained from any source, including biological and environmental samples. Biological samples may be obtained from animals (including humans) and encompass fluids, solids, tissues, and gases. Biological samples include blood products (e.g., plasma and serum), saliva, urine, and the like and includes substances from plants and microorganisms. Environmental samples include environmental material such as surface matter, soil, water, and industrial samples. These examples are not to be construed as limiting the sample types applicable to the present invention.

### DETAILED DESCRIPTION OF THE INVENTION

In some embodiments, the present invention provides mulitcomponent nanorods comprising two or more binding domains. In some embodiments, the binding domains comprise metals that serve as binding domains for protein affinity tags. In preferred embodiments, the nanorods are magnetic. In some embodiments, the present invention provides methods of purifying proteins or other molecules of interest using the nanorods. In some embodiments, two or more proteins of interest are fused to protein affinity tags that are specific for different binding domains of the nanorods. The fusion proteins are contacted with the nanorods, the nanorods are separated from the rest of the solution using a magnetic field, and the fusion proteins are selectively eluted from the nanorods. By altering the solution conditions, the fusion proteins can be separately eluted from the nanorods, allowing for the simultaneous purification of two or more proteins of interest. The present invention thus provides improved methods of purifying proteins.

Nanomaterials have been used extensively in the development of high sensitivity and high selectivity biodetection schemes. (Nam et al., Science 2003, 301:1884; Cao et al., Science 2002, 297:1536; Park et al., Science 2002, 295:1503; Cui et al., Science 2001, 293:1289). Designer particles, including noble metal polyhedral structures, quantum dots, nanopatterns, and nanorods have found application in many forms of biological tagging schemes, including DNA and protein detection, cell sorting, and histochemical staining (Bauer et al., Langmuir 2003, 19:7043; Birenbaum et al., Langmuir 2003, 19:9580; Salem et al., Nat. Mater. 2003, 2:668; Gu et al., J. Am. Chem. Soc. 2003, 125:15702; Penn et al. Curr. Opin. Chem. Biol. 2003, 7:609; Bruchez et al., Science 1998, 281:2013; Keating et al., Adv. Mater. 2003, 15:451; Caswell et al., 2003, 125:13914). Significant advantages over conventional molecule-based fluorophore strategies have been identified for several of these structures (Reiss et al., J. Electroanal. Chem. 2002, 522:95; Storhoffet al., J. Am. Chem. Soc. 1998, 120:1959; Hultgren et al., J. Appl. Phys. 2003, 93:7554). Other applications for nanomaterials in biology, beyond diagnostics, include therapeutics and separations (Penn et al., supra).

A key area for researchers working with proteins involves separation and purification. Traditionally, nickel columns have been used in conjunction with histidine tagged proteins to separate such structures from a matrix of other undesirable biological elements. Accordingly, in some embodiments, the present invention provides improved supports for such separations in the forms of nanorod structures (e.g., prepared by the porous template synthesis approach pioneered by Martin and Moskovits) (Martin, Science 1994, 266:1961; Routkevitch et al., J. Phys. Chem. 1996, 100:14037). This synthetic procedure allows one to prepare rods electrochemically with uniform diameters and with predefined block lengths of inorganic and organic materials with excellent control (Park et al., Science 2004, 303:348; Nicewarner-Pena et al., Science 2001, 294:137).

Experiments conducted during the course of development of the present invention demonstrated how one can use multi-component triblock rod structures for the immobilization of proteins and/or oligonucleotides and more particularly, for the use of immobilized proteins and/or oligonucleotides in effecting separations and/or purifications. In some embodiments, the rods have first segments (e.g., Ni) and a second segment (e.g., gold) as materials that can very efficiently separate his-tagged proteins from non-his tagged structures. The study demonstrated a novel approach to separating biomolecules utilizing multicomponent gold-nickel nanorods. The multicomponent rods can act as a high surface area scaffolding for biospecific recognition events that can be used in a variety of bioseparation schemes. The magnetic Ni block not only provides specificity with respect to His-tagged proteins, but also allows one to conveniently remove rods from a solution by use of a magnetic field. This approach to separation, which relies on designer nanomaterials as scaffolds, provides a versatile and convenient alternative to more cumbersome Ni column chromatography methods for separating multicomponent protein-containing mixtures.

I. Nanorods

In some embodiments, the present invention provides nanorods for use in protein purification. Nanorods may be fabricated using any suitable method, including, but not limited to, those disclosed herein (See e.g., Examples 1 and 2). In some embodiments, the nanorods comprise a core material coated with a function material. In preferred embodiments, the functional material is a metal that serves as a binding domain for a protein affinity tag or as an attachment point for further functionalization. The present invention is not limited to a particular metal for functionalization of nanorods. In some preferred embodiments, nickel and gold find use with the methods of the present invention. However, any metal that serves to functionalize a nanorod may be utilized. Other suitable metals may be utilized including, but not limited to, silver, silicon, copper, platinum, carbon, zinc, cobalt, aluminum, etc.

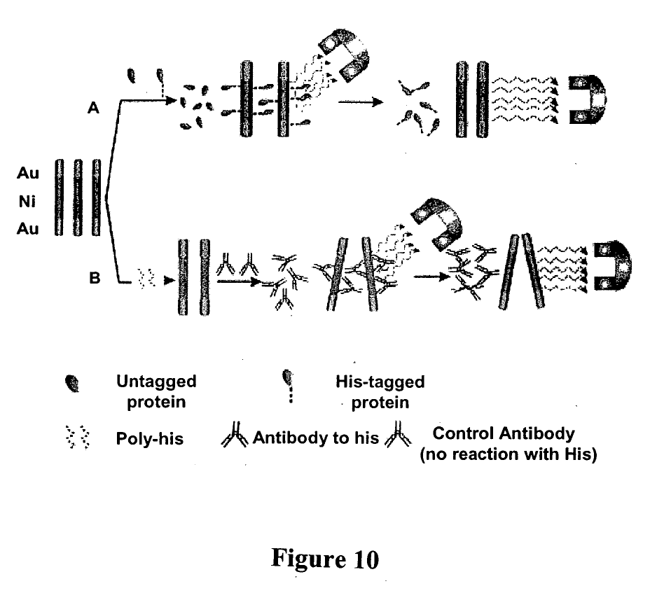

In some preferred embodiments, nanorods of the present invention are multifunctional. In some embodiments, multifunctionality is obtained by functionalizing different segments of the nanorod with different materials. For example, in some embodiments, the nanorods comprise two or more (e.g., three or more) binding domains or segments of the nanorod. Each of the segments comprises a different metal. One exemplary nanorod of the present invention is diagramed in FIG. 10. The present invention is not limited to the three component nanorod described herein. Additional segments (e.g. comprising additional functional groups) may be added to the nanorods to provide expanded functionality.

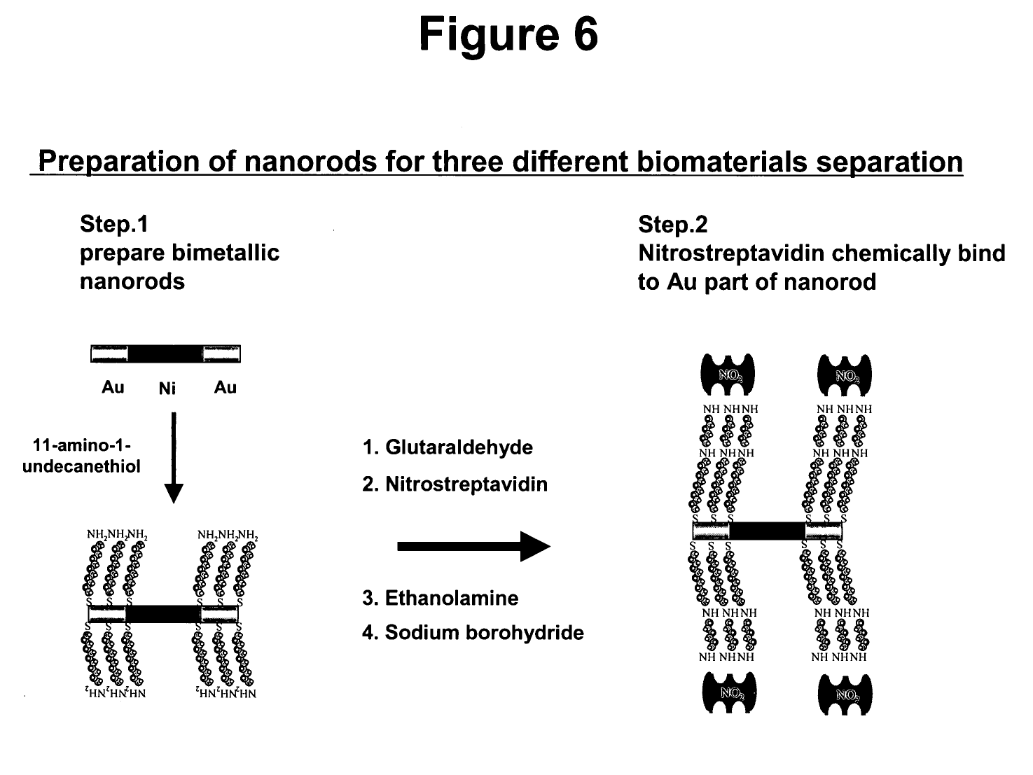

In some embodiments, metal nanorods are further functionalized. In some embodiments, nanorods are further functionalized to comprise binding domains for protein affinity tags. One exemplary method of functionalizing gold nanorod segments with nitro streptavidin is shown in FIG. 6. The present invention is not limited to the functionalization methods described herein. One skilled in the relevant art recognizes that additional chemistries may be utilized for attachment of other functional groups. For example, in some embodiments, linkers are used to attached functional groups (e.g., a 40 atom linker with a low negative charge density as described in (Shchepinov et al., Nucleic Acids Research 25: 1155 [1997]). In other embodiments, nanorods are functionalized with antibodies (e.g., specific for a protein or other molecules of interest). Additional methods for functionalizing surfaces for the attachment of molecules are described in U.S. Pat. Nos. 6,689,858, and 6,569,979, herein incorporated by reference in their entireties.

In some embodiments, the nanorods are magnetic. Thus, an appropriately applied magnetic field is used to effect separation of the protein-rod complex from a multicomponent solution. The separation of nanorods by magnetic fields provides a simple, inexpensive method of separating the nanorods (e.g., bound to a protein of interest) from a cellular lysate.

The present invention further provides nanorods on solid surfaces. For example, in some embodiments, nanorods are attached to columns, minicolumns (e.g. DARAS, Tepnel, Cheshire, England), HydroGel (Packard Instrument Company, Meriden, Conn.), fiber optic bundles, slides, capillaries, multiwell plates and other solid supports.

II. Protein Purification Using Nanorods

In some embodiments, the present invention provides methods of purifying proteins and other molecules (e.g., antibodies, ligands, lipids, nucleic acids, etc.) utilizing multifunctional nanorods. In preferred embodiments, the methods of present invention exploit binding interactions between protein affinity tags and functional groups on nanorods.

In some embodiments, a fusion protein between the protein of interest and a protein affinity tag is constructed used known recombinant DNA technology. The resulting fusion protein is preferably contained in an expression vector for overexpression of the fusion protein comprising the protein of interest. Vectors for generation of fusion proteins are commercially available.

In some embodiments, the present invention provides methods of separating a single protein of interest from a solution using a multifunctional nanorod. For example, in some embodiments, the gold-nickel-gold nanorods described in example 1 are utilized to separate histidine tagged proteins of interest. The gold component in such nanorods serves to block non-specific binding of proteins other than the protein of interest. The protein of interest, expressed as a fusion protein with histidine, preferentially binds to the nickel domain of the nanorods. A magnetic field is used to separate the nanorods and any protein bound thereto from the solution. The nanorods are washed to remove any non-specifically bound proteins, and the protein of interest is removed using appropriate solution conditions.

In other embodiments, the present invention provides methods of utilizing multifunction nanorods for the concurrent separation of two differently tagged proteins. For example, in some embodiments, nickel sections of multifunction nanorods bind with histidine labeled proteins and gold sections of multifunctional nanorods are further functionalized with nitrated streptavidin and bind to biotin tagged proteins.

In bioseparation methods, histidine-Ni interaction and streptavidin-biotin interaction are common tools for purification of proteins. The high affinity and specificity of streptavidin for biotin allows purification of the biotinylated protein under high stringency conditions, reducing background binding observed with other affinity tags because there are very few naturally biotinylated proteins. In some embodiments, the Au portions of Au/Ni/Au structured nanorods are selectively modified with streptavidin using 11-amino-1-undecanethiol and glutaraldehyde. Histidine is then attached to the Ni portion of the nanorod. In preferred embodiments, the biotin attached proteins and the histidine-attached proteins are then selectively released by different elution conditions.

### EXPERIMENTAL

The following examples serve to illustrate certain preferred embodiments and aspects of the present invention and are not to be construed as limiting the scope thereof.

### Example 1

Magnetic multi-segment nanorods composed of nickel and gold blocks were synthesized using electrodeposition into a porous alumina membrane. A thin layer of silver (200 nm) was evaporated on one side of an alumina filter (Whatman International Ltd, d=13 mm, pore size=20 nm; the pore diameter in the central region of the filter is substantially larger than the quoted 20 nm) and served as a cathode in a three electrode electrochemical cell after making physical contact with aluminum foil. Platinum wire was used as a counter electrode, and Ag/AgCl was used as the reference electrode. The nano-pores were partially filled with Ag, leaving headroom to accommodate the growth of additional domains (Technic ACR silver RTU solution from Technic, Inc.) at a constant potential, −0.9 V vs Ag/AgCl, by passing 1.5 C/cm₂ for 30 min. An Au block was then electroplated from Orotemp 24 RTU solution (Technic, Inc.) at 0.9 V vs Ag/AgCl followed by a Ni block from nickel sulfamate RTU solution (Tecnnic Inc.) at −0.9 V vs Ag/AgCl. The procedure involving gold was repeated to form the third and final capping block. Each segment length was controlled by monitoring the charge passed through the membrane. The first 1.4 μm (±0.2) long block of gold was generated by passing 1.3 coulombs. The 7.9 μm (±0.4) block of Ni required 15.4 coulombs, and the final 2.6 μm (±0.2) gold block involved the passing of 2.3 coulombs (the exposed membrane surface area is ˜1 cm²). The Ag backing and alumina membrane were dissolved with concentrated nitric acid and 3 M sodium hydroxide solutions, respectively. The gold portions of the three component structure were used to prevent nickel domain etching during the silver dissolving procedure. Prior to use, the rods were repeatedly rinsed with distilled water until the pH of the solution was 7. Nanorods containing more than three domains are prepared by repeating the above steps until the desired number of domains have been constructed. These added domains may be constructed of the same or different materials than used in the construction of the initial three domains, by appropriate selection of plating materials and conditions in the manner known to those skilled in the art.

The multicomponent nanorods were washed with methanol and ethanol to remove contaminants from their surfaces. This was done by suspending the nanorods in the desired solvent and using a magnetic field (BioMag, Polysciences, Inc.) to pull the rods to the sidewalls of a plastic vial while rinsing them with the appropriate solvent. The gold portions of the nanorods were passivated with 11-mercaptoundecyl-tri(ethylene glycol) (PEG-SH) by incubating the rod samples in 1 mL, 10 mM ethanolic solution of the surfactant for 2 hr followed by copious rinsing with ethanol and then Nanopure (Barnstead International, Dubuque, Iowa, USA) water.

Others have shown that alkylthiols preferentially modify the gold surface in such two component structures. The gold surface was modified with thiolated poly-(ethylene glycol) (PEG-SH) for two reasons. First, the PEG-SH minimizes nonspecific binding of proteins to the nanorod structures (Lopez et al., J. Am. Chem. Soc. 1993, 115:10774; Lee et al., Science 2002, 295:1702). Second, it stabilizes the rods by minimizing bare gold surface-surface interactions.

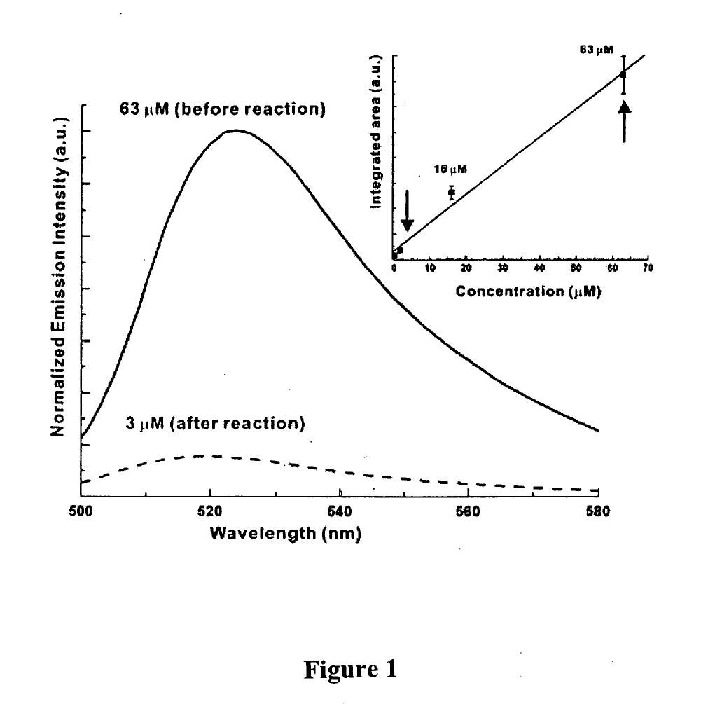

Poly-His was mixed with nanorods. The specific interaction between His and Ni blocks forms Poly-His tagged Au—Ni—Au rods. The specific interaction of polyhistidine (His ×6) with bulk oxidized nickel surfaces is known (Zhu et al., Science 2001, 293:2101). Similarly, fluorescein-tagged Poly-His (His ×6) binds specifically to the Ni portions of the nanorod as evidenced by confocal fluorescence microscopy. Au—Ni—Au nanorods (10⁹˜10¹⁰) were incubated in a 63 μM fluorescein labeled poly-His solution (1 mL, 0.1 M PBS (phosphate buffered saline), pH 7.4) for 12 hr at room temperature (22° C.). Then, the nanorods were vigorously rinsed with phosphate buffered saline (PBS) solution followed by Nanopure water. During each rinsing step, the rods were separated from the supernatant using magnetic force. Fluorescence imaging shows that the fluorescein-tagged polyhistidines efficiently bind to the Ni domains of the nanorod structures. This reaction between the poly-His and Au—Ni—Au rods can be monitored with the naked eye by watching the color of the solution decrease in intensity as a function of reaction time. A quantitative analysis of the efficiency of polyHis adsorption was performed by preparing a standard calibration curve from the fluorophore-labeled poly-His over a range of concentrations starting with the experimental poly-His initial concentration of 63 μM and going to 0.16 μM (inset FIG. 1).

The fluorescence emission intensity of supernatant solution isolated from the reacted nickel nanorods shows that ˜90% of the poly-His was captured by the rods from the starting solution, FIG. 1. As a control experiment, pure Au nanorods (no Ni), passivated with PEG-SH, were incubated in the poly-His solution (63 mM His in 0.1M PBS, pH 7.4) under nearly identical conditions and little interaction between the poly-His molecules and PEG-SH modified Au particles was observed (i.e. no detectable change in emission of the fluorescein as measured by fluorescence spectroscopy).

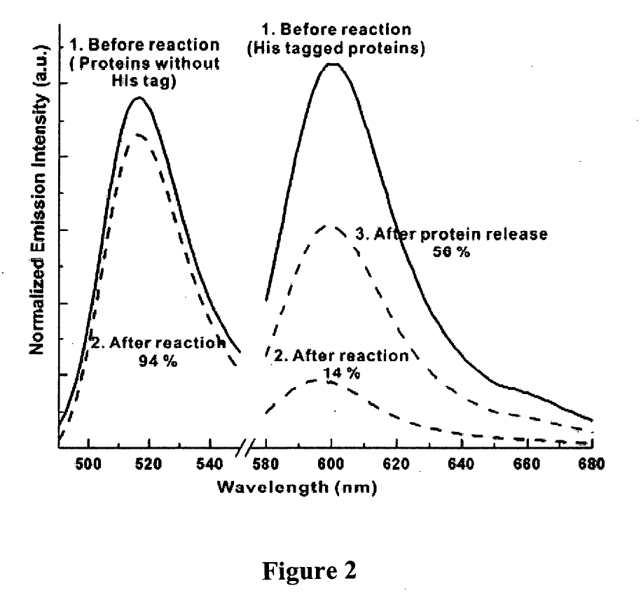

The Au—Ni—Au rods were further used in a novel scheme for separating His-tagged proteins from protein and other components without His Tags, FIG. 10A. For example, a 1 mL solution of two different proteins with different dye labels (anti-rabbit IgG without a His tag but labeled with Alexa 488 and His-tagged ubiquitin labeled with Alexa 568; concentration of each protein=100 μg/mL, in 0.1 M PBS, pH=7.4) were prepared. The orange solution containing the mixture of proteins was added to a vial containing PEG passivated nanorods (109˜1010). The solution was mechanically shaken for 24 hr. Application of a magnetic field caused the nanorods to move to the side walls, and the resulting green supernatant was collected and studied by fluorescence spectroscopy. Eluent buffer (pH 2.8, Pierce Biotech., Inc.) was added to the vessel containing the nanorods coated with His tagged, Alexa 568-labeled ubiquitin. This results in the release of the his-tagged proteins from the nanorods and formation of a red supernatant. Each of the solutions above were studied by UV-vis and fluorescence spectroscopy and compared with each other. The beginning mixture of proteins shows two bands at λₘₐₓ=516 nm and 600 nm, for each of the dye-labels. After adding the nanorods, there is an 86% decrease in the signal (λₘₐₓ=600 nm) for the dye-label associated with the His-tagged protein and only 6% for the protein without the His tag (λₘₐₓ=516 nm). After adding the eluent buffer, which is at a pH (=2.8) that results in release of the proteins from the Ni surface, a strong signal at λₘₐₓ=600 nm associated with the His tagged proteins was observed. Fluorescence spectroscopy shows that 56% of the original His-tagged ubiquitin has been effectively separated from the two component mixture in pure form (FIG. 2).

Finally, Poly-His tagged Au—Ni—Au rods are bioactive and selectively react with antibodies for poly-His, FIG. 10B. When a mixture of Alexa-488-labeled anti-human IgG and Alexa-568-labeled anti-polyHis IgG (100 μ/mL in 0.1 M PBS, pH 7.4) was introduced to a PBS solution (pH 7.4) of the Poly-His tagged Au—Ni—Au rods, the dye-labeled anti-poly-His IgG is very efficiently removed from the solution as evidenced by fluorescence spectroscopy. After 24 hr, 70% of the anti-polyHis (λₘₐₓ=600 nm) is removed from the solution while only 4 s of the signal (λₘₐₓ=516 nm) associated with the anti-Human IgG is lost. The rods can be attracted to the side of the reaction vessel with a magnetic field allowing one to remove the supernatant. The anti-poly-His can be released from the rods by adding eluent buffer at pH 2.8. Acidic conditions are known to decrease the specific interaction between the antibody and poly-His.

This Example demonstrates that Ni-containing nanorods can be used as novel materials for the efficient separation of mixtures of biomolecules by exploiting the chemical and physical properties of these nanostructures.

### Example 2

Use of Gold Functional Groups for Separations

This Example describes the use of Au portions of multifunctional nanorods as a docking site for biotin tagged proteins.

The general schematic illustration of in vivo biotinylated protein expression and purification is shown in FIG. 6. E. coli cells containing expression vectors such as Pinpoint vector express fusion proteins which have a tag for biotinylation of a protein of interest. The fusion protein is in vivo biotinylated by BirA biotin ligase in E. coli cells. The E. coli cells are lysed in a buffer compatible with the activity of the protein of interest and cellular debris is removed by centrifugation. The supernatant is applied slowly to the streptavidin resin or column to allow binding of the biotinylated protein. The resin or column is washed to remove nonspecifically bound protein. After washing, the biotinylated protein is eluted with a buffer containing high concentration free biotin, the eluate is collected. Finally, the biotinylated tag is removed by endoproteinase.

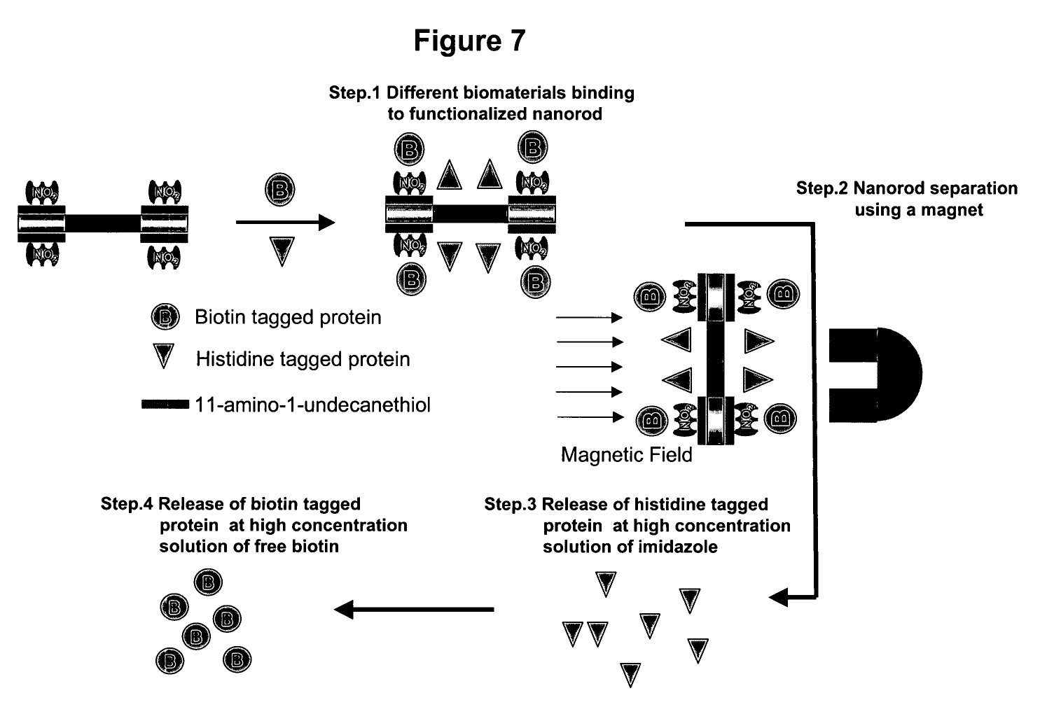

Schematic diagrams for the preparation and use of multifunction nanorods is shown in FIGS. 6 and 7. The Au portion of Au/Ni/Au structured nanorods was modified with 11-amino-1-undecanethiol by a self-assembly method. The primary amine groups on the surface of Au portion were activated by 8% glutaraldehyde, allowing amine groups of nitrated streptavidin to be covalently attached. A streptavidin sample (2.5 mg in 1 ml of 50 mM Tris buffer, pH 8) was treated with 50 mM of tetranitromethane for 50 min at room temperature. The sample was dialysed overnight to remove unreacted tetranitromethane with 1 M NaCl and deionized distilled water consecutively.

The nitrated streptavidin was attached on the Au portion of the nanorod by the incubation of the activated nanorod in a solution of nitrated streptavidin at a concentration of 1 ml of 1 uM nitrated streptavidin per ˜109 nanorods in PBS buffer at pH 7.4. The residual amine group of 11-amino-1-undecanethiol was inactivated with 0.2M of ethanolamine.

The nanorods were suspended in a mixture of biotin tagged protein and histidine tagged protein. The protein-loaded nanorods were then separated from the mixture by application of a magnetic field. A magnetic holder from Polysciences, Inc. was utilized. The biotin tagged proteins and histidine tagged proteins were released from the nanorods by different elution buffers.

SEM images of Au/Ni/Au structured nanorods were obtained by functionalizing Au blocks of Au/Ni/Au structured nanorod with streptavidin as described above. Green dye (fluorescein) labeled polyhistidine and red dye (Atto 590) labeled biotin were selectively bound on Ni block and Au ends of nanorods, respectively. Optical image and fluorescence images were obtained and show that polyhistidine was bound on the Ni blocks and biotin was bound on Au blocks of the nanorods.

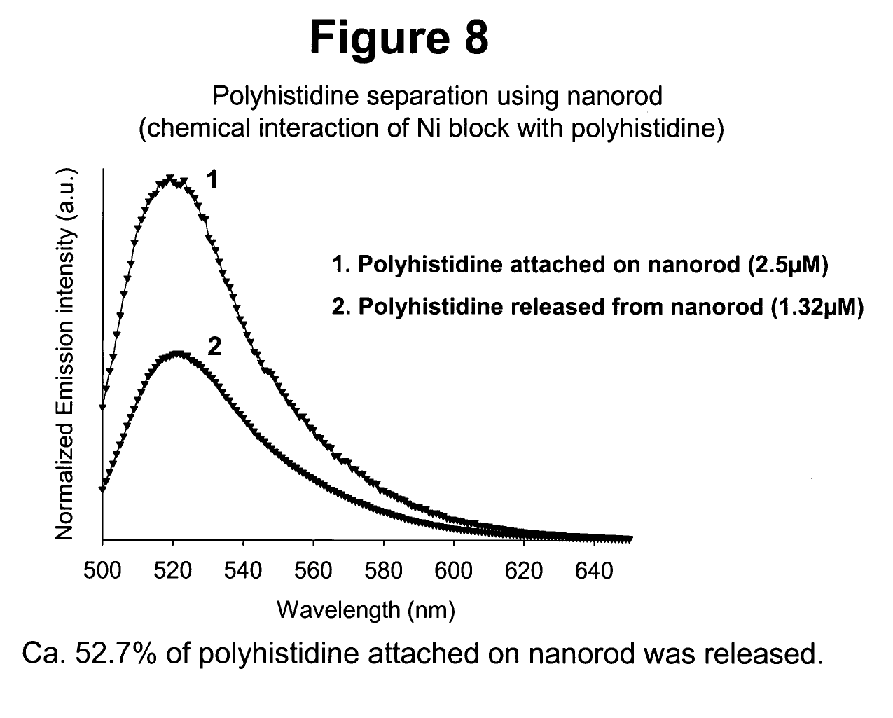

Au/Ni/Au nanorods (˜10¹⁰) functionalized with nitrated streptavidin as described above were incubated in a fluorescein labeled polyhistidine solution and then fluorescein labeled polyhistidines were released from the nanorods using elution buffer (>200 mM imidazole solution in PBS). Fluorescence spectrum of attached and released polyhistidine is shown in FIG. 8. A decrease in fluorescence upon release of polyhistidine is evident.

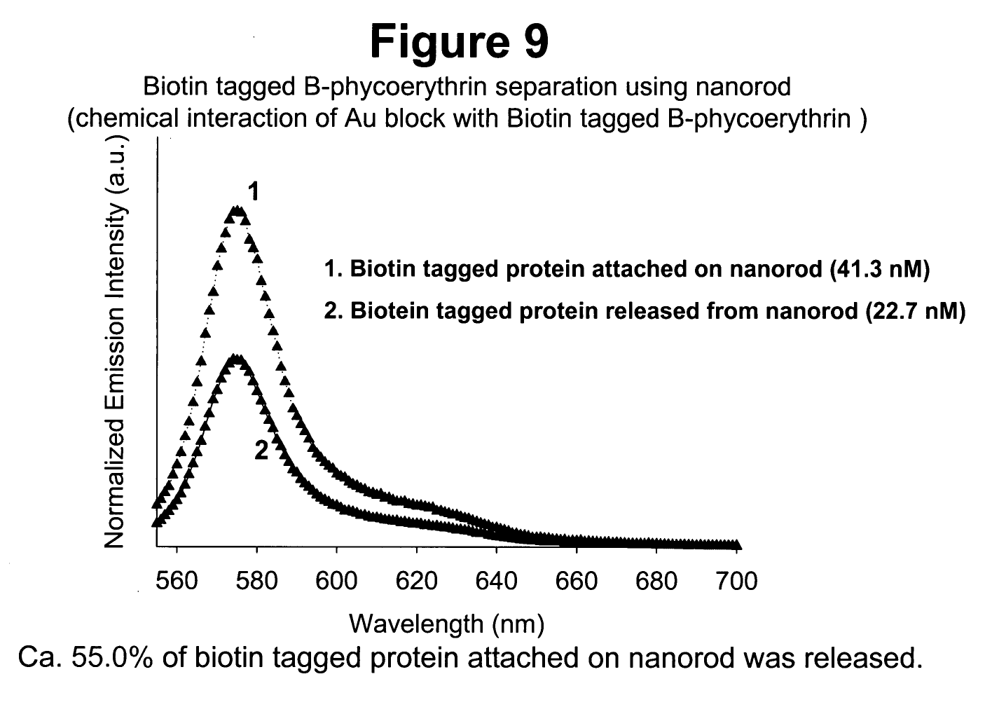

Au/Ni/Au nanorods (˜10¹⁰) functionalized with nitrated streptavidin as described above were incubated in a biotin tagged B-phycoerythrin solution. The protein was released using an elution buffer of >2 mM biotin solution in PBS. The results are shown in FIG. 9. The fluorescence spectrum of biotin tagged B-phycoerythrin attached on Au ends of the nanorods is altered. 55% of biotin tagged B-phycoerythrin from the rod-attached B-phycoerythrin was recovered after releasing B-phycoerythrin from Au blocks of the nanorods.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the art are intended to be within the scope of the following claims.

1. A composition comprising a plurality of multifunctional nanorods, wherein said nanorods comprise one or more binding domains, and wherein said one or more binding domains are each configured to bind a protein affinity tag.

2. The composition of claim 1, wherein said one or more binding domains comprise two or more binding domains.

3. The composition of claim 2, wherein said one or more binding domains comprise a first binding domain that is configured to bind a first protein affinity tag and a second binding domain that is configured to bind a second protein affinity tag.

4. The composition of claim 3, wherein said first binding domain comprises nickel.

5. The composition of claim 3, wherein said second binding domain comprises gold.

6. The composition of claim 1, wherein said protein affinity tags are covalently attached to proteins of interest.

7. The composition of claim 3, wherein said first protein affinity tag is covalently attached to a first protein of interest and a second protein affinity tag is covalently attached to a second protein of interest, and wherein said first and second proteins of interest are different from each other and said first and second protein affinity tags are different from each other.

8. The composition of claim 1, wherein said nanorods are configured to be attracted to a magnetic field.

9. A method, comprising:
a) providing a plurality of multifunctional nanorods, wherein said nanorods comprise one or more binding domains, and wherein said one or more binding domains are each configured to bind a protein affinity tag; and
b) contacting said nanorods with protein affinity tags under conditions such that said protein affinity tags binds to binding domains of said nanorods.

10. The method of claim 9, wherein said one or more binding domains comprise two or more binding domains, and wherein said two or more binding domains comprise a first binding domain that is configured to bind a first protein affinity tag and a second binding domain that is configured to bind a second protein affinity tag.

11. The method of claim 10, further comprising the steps of c) eluting said first protein affinity tag from said nanorod under first conditions such that said second protein affinity tag is not eluted from said nanorod; and d) eluting said second protein affinity tag from said nanorod under second conditions.

12. The method of claim 10, wherein said first binding domain comprises nickel.

13. The method of claim 10, wherein said second binding domain comprises gold.

14. The method of claim 9, wherein said protein affinity tags are covalently attached to proteins of interest.

15. The method of claim 10, wherein said first protein affinity tag is covalently attached to a first protein of interest and said second protein affinity tag is covalently attached to a second protein of interest, and wherein said first and second proteins of interest are different from each other and wherein said first and second protein affinity tags are different from each other.

16. The method of claim 10, wherein said nanorods are configured to be attracted to a magnetic field.

17. A kit, comprising: a plurality of multifunctional nanorods, wherein said nanorods comprise one or more binding domains, and wherein said binding domains are configured to bind one or more protein affinity tags.

18. The kit of claim 17, wherein said multifunction nanorods comprise two or more binding domains, and wherein said two or more binding domains comprise a first binding domain and a second binding domain.

19. The kit of claim 18, wherein said first binding domain comprises nickel.

20. The kit of claim 18, wherein said second binding domain comprises gold.
